(19) United States
(12) Patent Application Publication
(10) Pub. No.: US 2018/0305401 A1
(43) Pub. Date: Oct. 25, 2018

(54) PROCESSES FOR PURIFYING PROTEINS FROM PLASMA

(71) Applicant: Cambryn Biologics, LLC, Sarasota, FL (US)

(72) Inventors: Joseph A. BUETTNER, Sarasota, FL (US); Saida JENNELL, Sarasota, FL (US); Anna Fomina LEVINE, Sarasota, FL (US)

(21) Appl. No.: 15/770,111

(22) PCT Filed: Oct. 21, 2016
(86) PCT No.: PCT/US2016/058191

§ 371 Date: Apr. 20, 2018

Related U.S. Application Data
(60) Provisional application No. 62/244,488, filed on Oct. 21, 2015.

(51) Int. Cl. C07K 1/22; C07K 1/18; C07K 14/81; C07K 16/06

(52) U.S. Cl. CPC C07K 1/22; C07K 1/18; C07K 2317/526; C07K 16/065; C07K 2317/21; C07K 14/8125

(57) ABSTRACT

The invention provides processes for producing preparations (e.g., plasma preparations or fibrinogen (Fg)-depleted preparations) containing one or more proteins (e.g., plasma proteins). Processes of the invention can be used to obtain enriched preparations of one or more proteins (e.g., Fg, immunoglobulin (Ig; e.g., IgG), alpha-1 proteinase inhibitor (A1 PI), albumin, plasminogen, prothrombin complex, and/or other plasma proteins). Multiple enriched preparations can be obtained from a single sample (e.g., a whole blood or plasma sample) using the processes of the invention.

### BACKGROUND OF THE INVENTION

Plasma contains numerous proteins that are indispensable for blood clotting pathways. In patients suffering from deficiencies in one or more of such clotting factors, plasma augmentation therapy can be an effective approach. However, infusion of compositions that are enriched in critical plasma proteins is superior in efficiency and efficacy.

Immunoglobulin G (IgG) is the most common antibody isotype in plasma, having a concentration of about seven grams per liter of plasma. Patients with poor IgG levels or IgG function suffer from various immunodeficiency-related diseases. These patients often benefit from prophylactic or therapeutic immunoglobulin augmentation therapy via intravenous, or IV, delivery (i.e., IVIG), which has become a standard treatment for such cases.

Another plasma protein of clinical interest is alpha-1 proteinase inhibitor (A1PI, also known as alpha-1 antitrypsin). A1PI functions to prevent the degradation of structurally critical proteins such as elastin, and deficiency in A1PI can lead to the development of diseases such as emphysema. Patients suffering from A1PI deficiency are treated with weekly intravenous administrations of purified A1PI.

Fibrinogen (Fg) is another plasma component of therapeutic interest. Fg augmentation therapy is indicated for treatment and prevention of hemorrhage in congenital and acquired Fg deficiency, i.e., hypofibrinogenaemia, which impairs clotting pathways. For this reason, Fg is often isolated from donor plasma. Because numerous purified plasma components are highly sought after, a process to sequentially purify each desired component with high efficiency is desirable.

Plasma proteins such as IgG, A1PI, and Fg that are used in intravenous augmentation therapy regimens are purified from whole plasma from donors by plasmaphoresis. Current large scale purification processes are generally based on the antiquated technique of Cohn fractionation, which is associated with several inherent drawbacks. Such processes generate several precipitation fractions by gradually increasing the concentration of ethanol in cold plasma samples at times, and by radically altering the pH at times. Ethanol can be damaging to protein structure, placing inherent limitations on the efficiencies of Cohn fractionation-based processes. For example, IgG yield from the classic Cohn fractionation scheme is less than 50% of total whole plasma IgG. There is a pressing need in the field for methods to more efficiently purify multiple plasma proteins on a large scale.

Additionally, since conventional processes rely solely on differential solubilities of plasma proteins based on pH, ethanol concentration, temperature, ionic strength, and protein concentration, they lack specificity between proteins having similar structure.

To overcome these limitations, there is a need in the field for scalable processes that efficiently produces enriched compositions of various plasma proteins from a single source of whole plasma.

### SUMMARY OF THE INVENTION

The invention provides improved and scalable processes for producing multiple proteins from a single plasma source. In one aspect, the invention features a process for producing a Fg-depleted preparation enriched in IgG, including the steps of: contacting Fg-depleted plasma with an affinity chromatography adsorbing agent that binds IgG to produce adsorbed IgG and a solution including non-adsorbed proteins; separating the solution including non-adsorbed proteins from the adsorbed IgG; and eluting adsorbed IgG from the adsorbing agent to produce a Fg-depleted preparation enriched in IgG.

In one embodiment, the affinity chromatography adsorbing agent binds to a CH3 domain, e.g., a CH3 domain of a human Immunoglobulin (e.g., IgG, e.g., IgG1, IgG2, IgG3, or IgG4).

In one embodiment, the process further includes the steps of: passing the Fg-depleted preparation enriched in IgG through one or more ion exchange columns to separate IgG from contaminates; recovering IgG from the ion exchange column to produce a purified Fg-depleted preparation enriched in IgG, diafiltering the Fg-depleted preparation of enriched IgG to produce a diafiltrate, and sterilizing the diafiltrate. In one embodiment, a portion of the contaminates bind the column. In another embodiment, a portion of the IgG binds the column.

In a separate aspect, the invention provides a process for producing a Fg-depleted preparation enriched in alpha-1 proteinase inhibitor (A1PI), including the steps of: contacting Fg-depleted plasma with an affinity chromatography adsorbing agent that binds A1PI to produce adsorbed A1PI and a solution including non-adsorbed proteins; separating the solution including non-adsorbed proteins from the adsorbed A1PI; eluting adsorbed A1PI from the adsorbing agent to produce a Fg-depleted preparation enriched in A1PI; the Fg-depleted preparation enriched in A1PI is passed through one or more ion exchange columns to separate the A1PI from contaminates and the A1PI is recovered from the ion exchange resin to produce a purified Fg-depleted preparation of enriched A1PI. In one embodiment, a portion of the contaminates bind the column. In another embodiment, a portion of the A1PI binds the column. In some embodiments, the Fg-depleted preparation in A1PI can be diafiltered to produce a diafiltrate. In other embodiments, the preparation can be diluted to lower the salt content.

In some embodiments, the process is preceded by diafiltering or diluting the Fg-depleted plasma. In some embodiments, the process is preceded by virally reducing the Fg-depleted plasma (e.g., by incubating the plasma sample with a viricide solvent/detergent solution in an amount and duration sufficient to inactivate lipid-containing viruses).

In another aspect, the invention provides a process for producing a Fg-depleted preparation enriched in prothrombin complex. The process includes precipitating proteins comprising prothrombin from Fg-depleted plasma by reducing the pH to a sufficient level (e.g., pH 5.3) to produce a prothrombin complex precipitate and a solution comprising non-precipitated proteins. The prothrombin complex precipitate can then be separated from the supernatant comprising the non-precipitated proteins, and the prothrombin complex precipitate can be dissolved to produce a Fg-depleted preparation enriched in prothrombin complex. In some embodiments, the process further includes depth filtering the Fg-depleted preparation of enriched prothrombin complex. In some embodiments, the resulting filtrate enriched for prothrombin complex is passed through one or more ion exchange columns to separate the prothrombin from contaminates, and the prothrombin complex is recovered to produce a purified Fg-depleted preparation enriched in prothrombin complex. Either the contaminates or the prothrombin complex can bind the column. In some cases, the column is an anion exchange column (e.g., having a diethy-aminoethyl (DEAE) resin), and the prothrombin complex is eluted off the column using a buffer comprising NaCl, e.g., 0.15 M NaCl. In some cases, the process further includes diafiltering the purified Fg-depleted preparation of enriched prothrombin complex to produce a diafiltrate, and sterilizing the diafiltrate to produce a sterilized composition.

In another aspect, the invention features a process for producing a Fg-depleted preparation enriched in albumin. In some cases, the process involves passing a Fg-depleted plasma preparation (e.g., the supernatant from a prothrombin-purification process as describe above) through one or more anion exchange columns to separate the albumin from contaminates, and recovering the flow-through fraction containing albumin. The pH of the flow-through fraction containing albumin can be raised (e.g., to pH 7.4), and the albumin-containing flow-through fraction can again be passed through one or more anion exchange columns to separate the albumin from contaminates. The flow-through from this exchange may be discarded and the albumin can be recovered from the column to produce a purified Fg-depleted preparation enriched in albumin (e.g., by eluting the albumin, e.g., using a 0.25 M NaCl buffer). The resulting preparation can be diafiltered and sterilized.

In another aspect, the invention provides a process for producing a Fg-depleted preparation enriched in a plasma protein of interest by first contacting the Fg-depleted plasma with an affinity chromatography adsorbing agent that binds the plasma protein of interest to produce adsorbed protein of interest and a solution including non-adsorbed proteins. Next, the process includes separating the solution including non-adsorbed proteins from the adsorbed protein of interest and eluting adsorbed protein of interest from the adsorbing agent to produce a Fg-depleted preparation enriched in a protein of interest. The final steps of this process include passing the Fg-depleted preparation enriched in a protein of interest through one or more ion exchange columns to separate the protein of interest from contaminates and recovering the protein of interest from the ion exchange resin to produce a Fg-depleted preparation enriched in a protein of interest. In one embodiment, a portion of the contaminates bind the column. In another embodiment, a portion of the protein of interest binds the column.

In another aspect, the invention provides a process for producing a Fg-depleted preparation enriched in a first plasma protein of interest and a Fg-depleted preparation enriched in a second plasma protein of interest. First, the process includes contacting Fg-depleted plasma with a first affinity chromatography adsorbing agent that binds the first plasma protein of interest to produce an adsorbed first protein of interest and a first solution including non-adsorbed proteins, separating the first solution including non-adsorbed proteins from the adsorbed first protein of interest, and eluting the adsorbed first protein of interest from the adsorbing agent to produce a Fg-depleted preparation enriched in the first plasma protein of interest. Next, the first solution having non-adsorbing proteins is contacted with a second affinity chromatography adsorbing agent that binds the second plasma protein of interest to produce an adsorbed second protein of interest and a second solution including non-adsorbed proteins. The second solution including non-adsorbed proteins is then separated from the adsorbed second protein of interest and the adsorbed second protein of interest is eluted from the second adsorbing agent to produce a Fg-depleted preparation enriched in the second plasma protein of interest. The preparation enriched in the first plasma protein of interest is then passed through one or more ion exchange columns to separate the first protein of interest from contaminates. The first protein of interest is then recovered from the ion exchange column to produce a purified Fg-depleted preparation enriched in the first protein of interest. The preparation enriched in the second plasma protein of interest is then passed through one or more ion exchange columns to separate the second protein of interest from contaminates. In some embodiments, a portion of the contaminates bind the column. In other embodiments, a portion of the proteins of interest bind the column. The process can be additionally include (or be concluded by) eluting the adsorbed second protein of interest from the ion exchange resin to produce a purified Fg-depleted preparation of enriched second protein of interest.

In some embodiments, the first or second plasma protein of interest is IgG and the other protein of interest is A1PI. In some embodiments, the first or second affinity chromatography agent binds to a CH3 domain, e.g., a CH3 domain of a human IgG1, IgG2, IgG3, or IgG4.

In some embodiments, the first or second ion exchange column includes a cationic resin. In some embodiments, the first or second ion exchange column includes an anionic (e.g. weak anionic) resin. In another aspect, the invention provides a process for producing a Fg-depleted preparation enriched in a plasma protein of interest. First, the proteins including Fg are precipitated from whole plasma by addition of a salt in sufficient quantity to produce a Fg precipitate and a solution having a plasma protein of interest. In some embodiments, the Fg precipitate contains plasminogen. In some embodiments, the Fg precipitate contains little or no plasminogen. Next, the Fg precipitate is separated from the supernatant having the plasma protein of interest and the supernatant having the plasma protein of interest is diafiltered or diluted. Next, the solution is contacted with an affinity chromatography adsorbing agent that binds the plasma protein of interest to produce adsorbed protein of interest and a solution including non-adsorbed proteins, and the solution including non-adsorbed proteins is separated from the adsorbed protein of interest and eluted from the adsorbing agent to produce a Fg-depleted preparation enriched in a plasma protein of interest. Next, the Fg-depleted preparation enriched in a protein of interest is contacted with an ion exchange resin produce protein of interest and a solution including non-protein of interest materials, and the protein of interest is recovered from the ion exchange resin to produce a purified Fg-depleted preparation enriched in a protein of interest. The Fg precipitate can be reconstituted in a suitable buffer to produce a Fg solution. Further, the Fg solution can be incubated with a viricide solvent/detergent solution in an amount sufficient to inactivate lipid-containing viruses. Next, the process includes precipitating proteins including Fg from the solution by addition of a salt in sufficient quantity to achieve a precipitate of Fg and recovering and diafiltering the precipitate.

In another aspect, the invention provides a process for producing a Fg-depleted preparation enriched in a first plasma protein of interest and a Fg-depleted preparation enriched in a second plasma protein of interest. This process includes first precipitating proteins including Fg from whole plasma by addition of a salt in sufficient quantity to produce a Fg precipitate and a supernatant including the first plasma protein of interest and the second plasma protein of interest. Next, the Fg precipitate is separated from the supernatant, and the supernatant is diafiltered or diluted. The Fg-depleted plasma is contacted with a first affinity chromatography adsorbing agent that binds the first plasma protein of interest to produce an adsorbed first protein of interest and a first solution including non-adsorbed proteins. Next, the first solution including non-adsorbed proteins is separated from the adsorbed first protein of interest, and the adsorbed first protein of interest is eluted from the adsorbing agent to produce a Fg-depleted preparation enriched in the first plasma protein of interest. Next, the first solution including non-adsorbing proteins is contacted with a second affinity chromatography adsorbing agent that binds the second plasma protein of interest to produce an adsorbed second protein of interest and a second solution including non-adsorbed proteins. The second solution including non-adsorbed proteins is separated from the adsorbed second protein of interest, and the adsorbed second protein of interest is eluted from the second adsorbing agent to produce a Fg-depleted preparation enriched in the second plasma protein of interest. Next, the preparation enriched in the first plasma protein of interest is passed through one or more ion exchange columns to separate the first plasma protein of interest from contaminates. The first protein of interest is recovered from the ion exchange column to produce a purified Fg-depleted preparation of enriched first protein of interest. In one embodiment, the preparation enriched in the second plasma protein of interest is passed through one or more ion exchange columns to separate the second protein of interest from contaminates, and the second protein of interest is recovered from the ion exchange column to produce a purified Fg-depleted preparation of enriched second protein of interest.

In another aspect, the invention features a process for producing Fg-depleted preparations enriched in a first protein of interest, a second protein of interest, a third protein of interest, and, optionally, a fourth protein of interest. The process involves producing Fg-depleted preparations enriched in a first protein of interest and a second protein of interest by any process described above. In some embodiments, a flow-through fraction from the purification of the first or second protein of interest can be used as a Fg-depleted preparation from which a third and/or a fourth protein can be enriched. In some cases, the process includes precipitating proteins comprising a third protein of interest from the second solution comprising non-adsorbed proteins by reducing the pH to a sufficient level to produce a precipitate comprising the third protein of interest and a solution comprising non-precipitated proteins. The third protein of interest can then be separated (e.g., by centrifugation) from the supernatant comprising the non-precipitated proteins, and the precipitate can be dissolved to produce a Fg-depleted preparation enriched in the third protein of interest. Any further purification, filtration, diafiltration, sterilization, or any other processes may be carried out on the enriched third protein of interest as desired. In some cases, a fourth protein of interest may be enriched from the supernatant. For example, the supernatant can be brought to a desired pH (e.g., pH 4.6) and passed through one or more anion exchange columns to separate the fourth protein of interest from contaminates, and the flow-through fraction containing the fourth protein of interest can be recovered from the anion exchange column. In this case, the column binds the contaminates. Next, the pH of the flow-through fraction containing the fourth protein of interest can be raised (e.g., to pH 7.4) and passed through one or more additional anion exchange columns to separate the fourth protein of interest from contaminates. In this case, the flow-through fraction can be waste, and the column can bind the fourth protein of interest. Accordingly, the fourth protein of interest can be recovered by elution, e.g., with a NaCl buffer (e.g., a 0.25 M NaCl buffer) to produce a purified Fg-depleted preparation enriched in the fourth protein of interest.

In any of the preceding processes, the third protein of interest may include prothrombin (e.g., as a prothrombin complex), and the fourth protein of interest can be albumin.

In one embodiment, the Fg precipitate is reconstituted in a suitable buffer to produce a Fg solution and incubated with a viricide solvent/detergent solution in an amount sufficient to inactivate lipid-containing viruses. In some embodiments, the proteins including Fg can then be precipitated from the solution by addition of a salt to produce a Fg precipitate and recovered, diafiltered or diluted, and sterilized.

In another aspect, the invention provides a process for producing a Fg-depleted preparation enriched in a plasma protein of interest. In this aspect, the proteins including Fg are precipitated from whole plasma by addition of a first salt in sufficient quantity to produce a Fg precipitate and a supernatant including the plasma protein of interest. The proteins including the plasma protein of interest are precipitated from the supernatant by addition of second salt in sufficient quantity to produce the protein of interest precipitate. Next, the protein of interest is reconstituted in a suitable buffer to produce a solution, which is contacted with an affinity chromatography adsorbing agent that binds the plasma protein of interest, producing adsorbed protein of interest and a solution including non-adsorbed proteins. The solution including non-adsorbed proteins is separated from the adsorbed protein of interest, and the adsorbed protein of interest is eluted from the adsorbing agent to produce a Fg-depleted preparation enriched in a plasma protein of interest.

In some embodiments of any of the preceding processes, the plasma protein of interest comprises antithrombin III, fibronectin, plasminogen, Factor VII, Factor VIII, Factor IX, Factor X, Factor XI, or Factor XIII. In some embodiments, one or both of the first and second plasma protein of interest is antithrombin III, fibronectin, plasminogen, Factor VII, Factor VIII, Factor IX, Factor X, Factor XI, or Factor XIII.

In some embodiments, the salt includes potassium acetate, or trisodium citrate.

As used herein, the term “about” means ±10% of the recited value.

As used herein, “Fg complex” refers to a plurality of proteins, including Fg, that are physically associated by covalent or non-covalent interactions. A Fg complex may or may not include associated plasminogen. As used herein, “plasminogen-associated Fg complex” refers to a Fg complex that is associated with plasminogen. As used herein, “plasminogen-free Fg complex” refers to a Fg complex that is depleted of plasminogen.

As used herein, “supernatant” refers to the liquid phase of a composition having precipitated material. In many cases, the supernatant is the liquid positioned above a pellet generated by centrifugation. Alternatively, as used herein, a supernatant can refer to the liquid phase of a composition wherein precipitated material is dispersed throughout the composition (i.e., wherein the precipitate has not been pelleted down).

### BRIEF DESCRIPTION OF THE DRAWINGS

The accompanying drawing further describes the invention.

FIG. 1 is a flow diagram showing exemplary process steps for the isolation of three plasma proteins in accordance with one embodiment of the invention.

FIG. 2 is a flow diagram showing exemplary process steps for the isolation of five plasma proteins in accordance with a second embodiment of the invention.

### DETAILED DESCRIPTION OF THE INVENTION

The invention features processes for producing various compositions of proteins from a single sample of plasma. Specifically, the invention features affinity chromatography steps which separate a non-adsorbed solution from an adsorbed plasma protein, enabling subsequent purification procedures on both fractions. Furthermore, these chromatography purification steps efficiently purify proteins of interest from plasma fractions that have been depleted of fibrinogen (Fg). Thus, the modular processes of the invention allow for sequential production of numerous plasma proteins of clinical value from the same plasma sample.

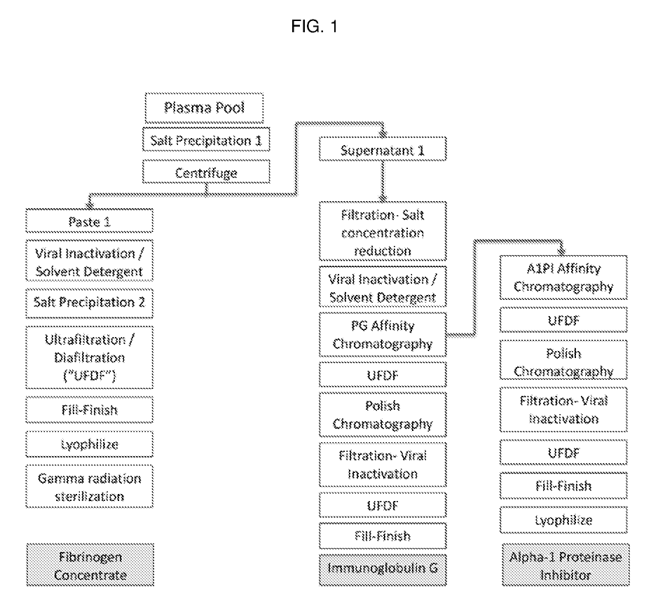

One exemplary process, shown by FIG. 1, includes purification of Fg, immunoglobulin G (IgG) and A1PI from plasmaphoresed human plasma. The Fg is precipitated out of the plasma solution by high concentrations of an organic salt (‘salting out’), a solvent/detergent (S/D) treatment reduces the virus load and after a second salt precipitation to remove the S/D the Fg is ready for formulation, sterile filtration, vial fill and lyophilization. The immunoglobins are purified from the Fg-depleted solution from the first salt precipitation by affinity chromatography. The purified IgG is polished with a cation or anion chromatography step, formulated, sterile filtered and vialed as a concentrated solution. The A1PI purification follows a similar design in that an affinity resin is utilized for the bulk of the purification as the ligand specificity is quite high. The starting material (SM) for the A1PI affinity chromatography is the flow-through (FT) from the IgG affinity run. The purified A1PI is polished with a strong anion chromatography step, formulated, sterile filtered, vialed and lyophilized. It is important to note that the affinity purifications can be performed in either order: either IgG first and A1PI second or A1PI first and IgG second. It makes no difference to the quality or yield of either product.

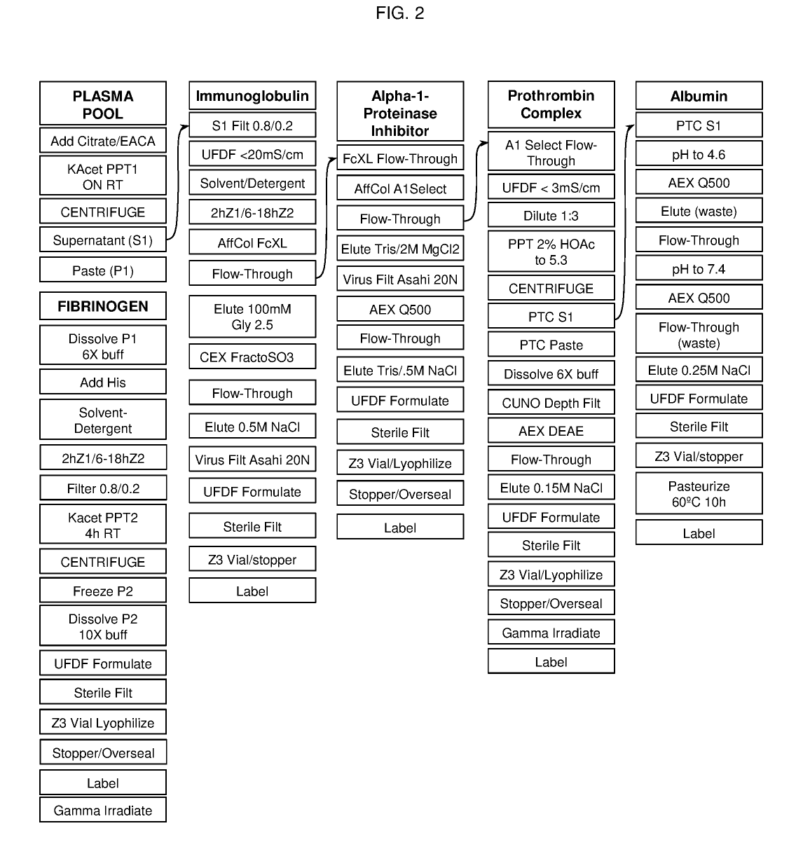

A second exemplary process, shown by FIG. 2, adds to the process of FIG. 1 by purifying additional proteins from the byproduct produced by the process of FIG. 1. In particular, the flow-through fraction of the affinity chromatography step of the A1PI isolation process can be used to isolate prothrombin (e.g., as part of a prothrombin complex) and albumin, as described herein.

Alternate procedures for some of the methods described herein are known by those skilled in the art, and the exemplary methods described herein are non-limiting.

### Pooling

Whole plasma can be collected by plasmaphoresis according to known methods.

In an exemplary method of the process, plasmaphoresed human plasma is then stored at −30° C. in a qualified freezer. A pick list is generated to identify the units required to create the pool (one or two units more than the necessary defined volume), then each unit is removed from the freezer and placed into a warm water bath to allow the plasma to thaw. The water bath has an initial temperature of 40° C. and, upon contact with the frozen unit, may rise in temperature up to 30° C. The temperature of the water batch is maintained from 30-40° C. for 1-2 hours. In an alternative embodiment, the units can be thawed in a 4° C. walk-in cooler for 60-68 hours. Once thawed, the plasma units are visually inspected for bottle integrity and hemolysis. Units found unacceptable are discarded and do not enter the pool. Each plasma bottle is sprayed with sterile 70% isopropanol to sanitize the lids and then the tops are either cut off or snapped off mechanically. Each plasma unit is swirled and emptied into a tared water-jacketed 316 stainless steel vessel with a single-use (sterile) processing bag and stirred to achieve uniformity until all plasma units have been pooled. The temperature of the vessel is set to 33° C. +/−3° C. Once the pooled plasma has reached the appropriate temperature it is mixed for 60 minutes (+/−15 minutes) to solubilize the cryo-precipitate. The net weight of the pool is notated and a sample is taken for testing and retain. At this point plasminogen is displaced off the Fg by addition of epsilon-amino caproic acid (EACA, granular, USP) in a ratio of 13.12 mg EACA per 1.0 KG solution, mixed at appropriate speed and held for 60 minutes +/−15 minutes. The net weight of the pool is notated and a sample is taken for testing and retain. Alternative methods for pooling plasma are known in the art.

### First Fg Precipitation

The invention features processes for producing a Fg-depleted preparation from whole plasma to be used as a source to produce additionally preparation of plasma proteins. Fg that is depleted from the whole plasma may be in the form of Fg complexes and may or may not include, e.g., plasminogen. In an exemplary method, the temperature of the pooled plasma is dropped to 20° C. and Potassium Acetate (KAcet, solution held at RT) is added until its final concentration is approximately 1.31 M KAcet. The precipitation solution (10 M Potassium Acetate, USP) is added at a ratio of 0.151 KG KAcet per 1.0 KG of plasma pool weight. The precipitation is allowed to complete by stirring the suspension for not less than 60 minutes. The suspension is centrifuged at 5,000-20,000×G with a residence time of at least 10 minutes in the bowl (note: bowl rotation speed depends upon pool scale). The clarified solution (Supernatant #1, Super 1) is decanted and the paste is harvested (Paste #1). Super 1 is transferred into a new bag in the water-jacket vessel. The net weight of the solution is notated and a sample is taken for testing and retain. One skilled in the art will understand that there are alternative methods to produce Fg-depleted plasma, e.g., by purifying Fg from whole plasma.

### Fg Product Line

The invention provides a process for producing a composition having a high concentration of Fg or complexes thereof. In another aspect of the invention, a plasma sample is depleted of Fg. Various methods for the removal of Fg from a plasma sample by preparing enriched Fg fractions are known in the art, and can be useful as part of the present invention.

In one method of Fg isolation, a salt-precipitation method is performed in which the Fg is further precipitated to remove additional contaminates. An exemplary method is described below.

### Paste 1 Dissolution

The paste from the KAcet precipitation is harvested from the centrifuge and dissolved in TBS-C (USP) buffer (50 mM Tris-HCl, 50 mM Na₃Citrate-2H₂O, 150 mM NaCl, pH=7.4) at ratio of 10 KG buffer per each 1.0 KG paste. The paste is dissolved at 40° C. +/−3° C. until the paste has been fully dissolved (as determined by, e.g., visual observation). Granular Histidine (USP) is added to the solution in a ratio of 0.8 g Histidine per 1.0 KG solution, mixed at appropriate speed and held for 10 minutes.

### Paste 1 S/D Treatment

All enveloped viruses and some non-enveloped viruses are denatured by addition of a two component chemical denaturant: the solvent TNBP (tri-n-butyl phosphate) and the detergent Tween 80 (Polysorbate 80). This solvent/detergent (S/D) treatment denatures the lipid membrane of enveloped rendering the virion uninfective. It is also reduces bacterial and fungal load and helps decrease endotoxin activity. The TNBP/Tween is mixed neat (at a 23.09:76.91 ratio of TNBP to Tween) to create the stock solution; then 13.2 g of the S/D stock is added to each 1.0 KG of paste 1 suspension for viral inactivation. The inactivation is allowed to proceed at least 6 hours (e.g., 6-18 hours, or overnight) at RT and appropriate mixing. After 2 hours of inactivation the solution is transferred into Zone 2 and allowed to continue until completion. The net weight of the solution is notated and a sample is taken for testing and retain.

### Second Fg Precipitation

Plasminogen is displaced off the Fg by addition of epsilon-amino caproic acid (EACA, granular, USP) in a ratio of 6.56 mg EACA per 1.0 KG solution, mixed at appropriate speed and held for 60 minutes +/−15 minutes at RT. The solution temperature is dropped to 1° C. +/−1° C., and then Potassium Acetate is added until its final concentration is approximately 1.31 M KAcet. The precipitation solution (10 M Potassium Acetate, USP) is added at a ratio of 0.151 KG KAcet per 1.0 KG of S/D solution weight. The precipitation is allowed to complete by stirring the suspension for 2-20 hours (e.g., 4 hours). The suspension is centrifuged at 5,000-20,000×G with a residence time of at least 10 minutes in the bowl (note: residence time depends upon bowl rotation speed, which depends upon pool scale). The clarified solution is a waste product and the paste is harvested (Paste #2).

### Paste 2 Dissolution

The paste from the second KAcet precipitation is harvested from the centrifuge. At this point, the paste may be frozen for storage. The paste can be dissolved in a Tris/NaCl/Citrate/His (USP) buffer (20 mM Tris-HCl, 150 mM NaCl, 20 mM Na₃Citrate-2H₂O, 80 mM Histidine, pH=7.4) until clottable Fg is within the target range of 25 mg/ml. A stabilizer (e.g., a sugar stabilizer) may optionally be added to an appropriate amount and the pH is adjusted, if necessary.

### Final Fg Sterility Filtration/Fill

The formulated Fg bulk is passed through a 0.2 μm sterile filter and transferred into the Filling Suite. 20-25 milliliters of sterile bulk is put into 50 ml critically-clean, sterile glass serum vials. Optionally, the Fg bulk is lyophilized and or/gamma irradiated. The vials are capped and sealed for storage. The resulting Fg concentrate yields about 84% of the whole plasma Fg concentration.

Purifying Proteins from Fg-Depleted Plasma

The invention provides a process for producing a composition having a high concentration of additional serum proteins of interest (e.g., IgG, A1PI, prothrombin complex, and/or albumin). This purification can be performed on a Fg-depleted plasma fraction (e.g., a supernatant from the previously described Fg precipitation process). Proteins of interest, including, but not limited to, IgG and A1PI, can be purified using affinity chromatography according to the exemplary methods provided below. It will be understood that these processes can be readily adapted (e.g., by substitution with a suitable affinity column) for purification of various other plasma proteins, including, but not limited to, antithrombin III, fibronectin, plasminogen, Factor VII, Factor VIII, Factor IX, Factor X, Factor XI, or Factor XIII, as described below.

Following purification of any preferred plasma protein by affinity chromatography, remaining plasma proteins, e.g., prothrombin complex and/or albumin, can then be purified according to processes described below. In general, albumin purification is reserved for the end of the process, preceded by prothrombin complex purification, e.g., according to the following exemplary methods.

### IgG Product Line

### Viral Reduction Filtration

Reduction of adventitious agents throughout the process is necessary to protect the product from degradation. A general filtration to clarify the solution and remove bacteria and fungi (spores and hyphae) is necessary to move the product from Pooling (Zone 0) into a similar volume vessel in Zone 1. Several filtration companies have tested their pre-filters and bioburden reduction filters for this particular stage of the process. It is important to note this filtration should not be considered a sterile filtration as the filters are an average (nominal) pore size and not absolute pore size. One skilled in the art will understand that there are alternative methods to reduce viral contaminates from plasma preparations (e.g., by gamma irradiation).

### Ultrafiltration/Diafiltration

A supernatant resulting from the Fg precipitation has high KAcet concentration which can be reduced prior to subsequent chromatography steps. Both the IgG and A1PI molecules are large enough that they do not pass through a 30 KD or a 50 KD diafilter membrane. In some cases, a 30 KD membrane may be used, but 50 KD membranes were chosen to maximize the flux of the system (flux is the amount of liquid flow through the membrane under various conditions). The UFDF system utilized for the 10 L plasma batch size reduces the conductivity from approximately 55-60 mS/cm to <20 mS/cm (e.g., approximately 5 mS/cm) with diafiltration against 3.6 volumes of water. This system runs about 30 L/Hr/m² which represents approximately 3.5 hours to diafilter the supernatant 1 (appropriate scaling of diafilter membrane size will keep the time constant). The target conductivity at this point can be below 10 mS/cm to allow binding in the first chromatography step. The solution passes over the 30 KD diafilter and when backpressure is supplied the solution will pass through the diafilter membrane carrying the low molecular weight compounds (<30 KD=salts, lipids, small proteins) to waste but retaining the proteins over 30 KD in size. The net weight of the solution is notated and a sample is taken for testing and retain. Alternatively, salt concentrations can be decreased using other methods known in the art, e.g., dilution in an appropriate buffer.

### Solvent/Detergent Virus Denaturation

An appropriate amount of the S/D stock (13.2 g S/D Stock to 1.0 KG solution) is added to the super 1 UFDF solution for viral inactivation. The inactivation is allowed to proceed for a period of time (e.g., at least 6 hours, e.g., 6-18 hours or overnight) at RT under appropriate mixing. After an acceptable period of time (e.g., 2 hours) of inactivation the solution is transferred into Zone 2 and allowed to continue until completion. The net weight of the solution is notated and a sample is taken for testing and retain. Alternative methods of S/D virus denaturation, as known in the art, can be performed.

### IgG Affinity Chromatography

Proteins of interest can be purified from a fibrinogen-depleted plasma preparation (e.g., by processes described above) using known affinity chromatography methods.

In an exemplary method, the affinity column ligand binds to a CH3 domain, e.g., a CH3 domain of a human IgG, e.g., IgG1, IgG2, IgG3, or IgG4. Such ligands include those included as part of the CAPTURESELECT™ FcXL Affinity Matrix (ThermoFisher Scientific). In some cases, the affinity column ligand is an IgG-binding cell surface receptor derived from Streptococcus bacteria and expressed through an E. coli system lacking the albumin binding domain. It is coupled onto a highly cross-linked Sepharose 4 Fast Flow resin. This resin is commercially available through GE Healthcare (cat #17-0618-05). The affinity column is run in a 50 mM Tris-HCl pH=7.4 solution. There is an optional 200 mM NaCl/Tris salt wash to disrupt non-specific ionic interactions, and the IgG is eluted off the column with low pH 100 mM Glycine-HCL at pH=2.50. The pH of the affinity column eluates (PGE) can be adjusted, depending on whether or not an anion exchange chromatography step will be subsequently performed. In cases in which polish chromatography involves anion exchange, the eluate is brought to neutral acidity by addition of the appropriate volume of a concentrated Tris solution. Alternatively, if cation exchange chromatography is performed, there may be no need to adjust the pH. The net weight of the solution is notated and a sample is taken for testing and retain.

### IgG Polish Chromatography

As discussed above, a polishing chromatography step can be used according to known methods to ensure no affinity ligand leaching from the resin ends up in the final container. In an exemplary method, ion-exchange chromatography (e.g., anion exchange chromatography or cation exchange chromatography) can be performed according to known methods. In some instances, cation chromatography exchange is performed, e.g., using a strong cation exchange resin (e.g., FRACTOGEL® EMD SO₃— (S)) according to conventional protocols or manufacturer instructions. The IgG can be eluted off the column with a salt. The net weight of the solution can be notated and a sample is taken for testing and retain.

### Viral Filtration

Following affinity chromatography (and, optionally polish chromatography as discussed above), viral filtration of the solution can be performed to reduce viral content. To prepare the virus filter (e.g., Asahi Planova 20N or Pall Pegasus SV) it can be rinsed with water for infusion (WFI) then tested to ensure the filter membrane is intact (i.e., no tears as this would render the virus filter ineffective). Integrity testing of the membrane uses an increase of air pressure to ensure no bubbles come through the membrane. This test can be performed before viral filtration and again after virus filtration to demonstrate full integrity of the filter and satisfy regulatory requirements.

The viral filtered eluate may contain high salt concentration, which can be removed, for example, by diafiltration or other acceptable method known in the art (e.g., simple dilution, or a UFDF system, as described herein). Further sterilization techniques can be performed on the final solution according to known methods. In some cases, the solution is sterile filtered. The net weight of the solution is notated and a sample is taken for testing and retain. The final yield of IgG can be up to about 80%. Vials containing the final IgG product are sealed and labeled.

### A1PI Product Line

### A1PI Affinity Chromatography

A1PI can be purified, e.g., from the flow-through fraction of the IgG affinity chromatography process, according to known methods (e.g., affinity chromatography). In one exemplary method, the affinity column ligand to bind A1PI is a single-domain antibody fragment (e.g., derived from llamas and expressed through a yeast system). It can be coupled, for example, onto a highly cross-linked agarose resin. This resin is commercially available through GE Healthcare (cat #17-5472-03). The affinity column is run in a 50 mM Tris-HCl pH=7.4 solution. There is a 150 mM NaCl/Tris salt wash to disrupt non-specific ionic interactions and the A1PI is eluted off the column with 2 M MgCl₂ in Tris buffer. The affinity column eluates (ACE) can be diafiltered to reduce the salt concentration. The net weight of the solution is notated and a sample is taken for testing and retain.

### Ultrafiltration/Diafiltration

A salt concentration of the resulting solution can be reduced by one of several known methods, including, e.g., simple dilution in an appropriate buffer or a UFDF system. In an exemplary method, a UFDF system is used. The UFDF system reduces the conductivity from approximately 120 mS/cm to approximately 5 mS/cm with diafiltration against 5 volumes of water. This system runs about 60 L/Hr/m² which represents approximately 0.5 hours to diafilter the ACE per batch. The target conductivity at this point needs to be below 10 mS/cm to allow binding in the second chromatography step. The net weight of the solution is notated and a sample is taken for testing and retain.

### Anion Exchange Chromatography

An anion exchange chromatography step can be used to remove any affinity ligand that may have leached from the resin. In one exemplary method, the Asahi Q500 anion exchange resin (AEX; cat#19907) can be used for the polishing step and the binding and elution parameters optimized for A1PI yield. The Q500 column is equilibrated in 50 mM Tris-HCl pH=7.4, and the A1PI is eluted with a step gradient to 350 mM or 500 mM NaCl in the Tris buffer. The net weight of the solution is notated and a sample is taken for testing and retain.

### Virus Filtration

Small non-enveloped viruses (e.g., parvoviruses) can be removed from the product by filtration through a small 20 nanometer pore. Larger non-enveloped viruses will also be removed with this step.

To prepare the virus filter (e.g., Asahi Planova 20N or Pall Pegasus SV4), it can be rinsed with water for infusion (WFI) then tested to ensure the filter membrane is intact (no tears as this would render the virus filter ineffective). Integrity testing of the membrane uses an increase of air pressure to ensure no bubbles come through the membrane. This test can be performed before viral filtration and again after virus filtration to demonstrate full integrity of the filter and satisfy regulatory requirements. The net weight of the solution is notated and a sample is taken for testing and retain. One skilled in the art will understand that there are alternative methods to effectively filter viral components from plasma preparations.

### UFDF Formulation Bulk

The viral filtered eluate can still contain high salt concentration, which can be removed, for example, by diafiltration or other acceptable method known in the art (e.g., simple dilution). The UFDF system reduces the conductivity from approximately 20 mS/cm to approximately 2 mS/cm with diafiltration against 3 volumes of water and allows buffer exchange into the Tris final formulation. This system runs about 60 L/Hr/m² which represents approximately 0.5 hours to diafilter the Q500 eluate per batch. The net weight of the solution is notated and a sample is taken for testing and retain.

### A1PI Sterility Filtration/Fill

A sterility filtration/fill step can be performed according to known methods. In one exemplary method, the formulated A1PI bulk is passed through a 0.2 μm sterile filter and transferred into the Filling Suite. Approximately 20 ml of sterile bulk is put into 50 ml critically-clean, sterile glass serum vials. In some cases, the A1PI is lyophilized. The final yield of A1PI can be about 80%. Vials containing the final A1PI product are sealed and labeled.

### Alternative Protein Product Line

### Affinity Chromatography

Various other plasma proteins are purified from a Fg-depleted plasma preparation using protein-specific affinity chromatography methods, such as those known in the art. Such plasma proteins include, but are not limited to, antithrombin III, fibronectin, plasminogen, Factor VII, Factor VIII, Factor IX, Factor X, Factor XI, and Factor XIII, any one or more of which (and, optionally, in addition to IgG and/or A1PI) can be purified in any sequence. Generally, any one or more of IgG, A1PI, antithrombin III, fibronectin, plasminogen, Factor VII, Factor VIII, Factor IX, Factor X, Factor XI, and Factor XIII (e.g., that are isolated by affinity chromatography) are purified from Fg-depleted plasma preparations, prior to any prothrombin complex and/or albumin purification.

Exemplary methods useful as part of the present invention for purification of antithrombin III by affinity chromatography are known in the art and described, for example, in Hoffman (The American Journal of Medicine, 87, S23-S26, 1989). Antithrombin III affinity chromatography resins are commercially available, e.g., CAPTURESELECT™ Antithrombin III Affinity Matrix (Thermo Fisher). Exemplary methods useful as part of the present invention for purification of fibronectin by affinity chromatography are known in the art and described, for example, in Kuyas et al. (Thrombosis and Haemostasis 63.3, 439-444, 1990). Exemplary methods useful as part of the present invention for purification of plasminogen by affinity chromatography are known in the art and described, for example, in U.S. Pat. No. 3,943,245 or Deutsch et al. (Science, 170, 1095-1096, 1970). Exemplary methods useful as part of the present invention for purification of Factor VII by affinity chromatography are known in the art and described, for example, in WO 2006/074664. Exemplary methods useful as part of the present invention for purification of Factor VIII by affinity chromatography are known in the art and described, for example, in U.S. Pat. No. 5,110,907. Exemplary methods useful as part of the present invention for purification of Factor IX and Factor X by affinity chromatography are known in the art and described, for example, in Ahmad et al. (Thrombosis Research, 55, 121-133, 1989). Factor IX affinity chromatography resins include, e.g., heparin-sepharose resins. Exemplary methods useful as part of the present invention for purification of Factor XI by affinity chromatography are known in the art and described, for example, in Mashiko et al. (Biol Chem Hoppe Seyler, 375, 481-484, 1994). Exemplary methods useful as part of the present invention for purification of Factor XIII by affinity chromatography are known in the art and described, for example, in U.S. Pat. No. 5,688,919 and De Backer-Royer et al. (International Journal of Biochemistry, 24, 91-97, 1992). It will be understood that any suitable affinity chromatography-based purification method of any one or more of IgG, A1PI, antithrombin III, fibronectin, plasminogen, Factor VII, Factor VIII, Factor IX, Factor X, Factor XI, and Factor XIII are contemplated for use as part of the present invention.

### Ultrafiltration/Diafiltration

A salt concentration of the affinity column eluate of any of the processes above can be reduced by one of several known methods, including, e.g., simple dilution in an appropriate buffer or a UFDF system. In an exemplary method, a UFDF system is used. The UFDF system reduces the conductivity from approximately 120 mS/cm to approximately 5 mS/cm with diafiltration against 5 volumes of water. This system runs about 60 L/Hr/m² which represents approximately 0.5 hours to diafilter the affinity column eluate per batch. The target conductivity at this point is below 10 mS/cm to allow binding in the second chromatography step. The net weight of the solution is notated and a sample is taken for testing and retain.

### Ion Exchange Chromatography

If necessary, ion exchange chromatography is performed as a polish chromatography step following affinity chromatography to remove any affinity ligand that may have leached from the resin of the affinity column in any of the processes described above. In some embodiments, cation exchange chromatography is used. In other embodiments, anion exchange chromatography is used. For either cation exchange or anion exchange, depending on the protein being purified, the binding and elution parameters are optimized for the specific protein according to methods known in the art. The protein can be eluted off the column with a salt (e.g., NaCl or other suitable salt) according to known methods. The net weight of the solution is notated and a sample is taken for testing and retain.

### Virus Filtration

Small non-enveloped viruses (e.g., parvoviruses) are removed from the product by filtration through a small 20 nanometer pore. Larger non-enveloped viruses are also removed with this step. To prepare the virus filter (e.g., Asahi Planova 20N or Pall Pegasus SV4), it can be rinsed with water for infusion (WFI) then tested to ensure the filter membrane is intact (no tears as this would render the virus filter ineffective). Integrity testing of the membrane uses an increase of air pressure to ensure no bubbles come through the membrane. This test can be performed before viral filtration and again after virus filtration to demonstrate full integrity of the filter and satisfy regulatory requirements. The net weight of the solution is notated and a sample is taken for testing and retain. One skilled in the art will understand that there are alternative methods to effectively filter viral components from plasma preparations or otherwise kill any contaminating virus (e.g., by gamma irradiation).

### UFDF Formulation Bulk

The viral filtered eluate can still contain high salt concentration, which can be removed, for example, by diafiltration or other acceptable method described above or known in the art (e.g., simple dilution). The net weight of the solution is notated and a sample is taken for testing and retain.

### Sterility Filtration/Fill

A sterility filtration/fill step can be performed according to methods known in the art or described herein. Vials containing the final protein product (antithrombin III, fibronectin, plasminogen, Factor VII, Factor VIII, Factor IX, Factor X, Factor XI, or Factor XIII) are sealed and labeled.

### Prothrombin Complex Product Line

A flow-through fraction of Fg-depleted plasma, e.g., the flow-through of the A1PI purification process, supra, can be further processed to isolate additional proteins, e.g., prothrombin complex, followed by albumin. A salt concentration of the previous flow-through fraction can be reduced by one of several known methods, including, e.g., simple dilution in an appropriate buffer or a UFDF system. In an exemplary method, a UFDF system is used. The UFDF system can reduce the conductivity from approximately 20 mS/cm to <4 mS/cm (e.g., <3 mS/cm) with diafiltration against 5 volumes of water. The resulting solution is diluted 1:3 and precipitated by bringing the pH to from about 7.4 to 5.3, e.g., using 2% acetic acid.

The prothrombin complex solution is then centrifuged according to methods known in the art or described supra to generate a prothrombin complex paste and a supernatant. The paste from the prothrombin complex precipitation is harvested from the centrifuge and dissolved in a buffer containing 20 mM Tris, 20 mM citrate, and 150 mM NaCl, pH 7.4 at 40° C. +/−3° C. until the paste has been fully dissolved (as determined by, e.g., visual observation).

Once dissolved, the prothrombin complex solution is purified using depth filtration according to methods known in the art. Suitable depth filtration systems include ZETA PLUS™ (3M).

An anion exchange chromatography step can be used to remove any affinity ligand that may have leached from the resin. In one exemplary method, a diethyl-aminoethyl (DEAE) anion exchange resin is optionally used and the binding and elution parameters optimized for prothrombin complex yield. The flow-through fraction may be retained for further purification, e.g., of albumin. The anion exchange column is equilibrated with 150 mM NaCl, and the prothrombin complex is eluted. The net weight of the solution is notated and a sample is taken for testing and retain.

The prothrombin complex eluate may contain a high salt concentration, which can be removed, for example, by diafiltration or other acceptable method known in the art (e.g., simple dilution or UFDF, according to methods known in the art or described supra). The net weight of the solution is notated and a sample is taken for testing and retain.

A sterility filtration/fill step can be performed according to methods known in the art or described herein. In one exemplary method, the formulated prothrombin complex bulk is passed through a 0.2 μm sterile filter and transferred into the Filling Suite. Approximately 20 ml of sterile bulk is put into 50 ml critically-clean, sterile glass serum vials. In some cases, the prothrombin complex is lyophilized. Vials containing the final prothrombin complex product are sealed and labeled. Additionally, the prothrombin complex product may be gamma irradiated.

### Albumin Product Line

A flow-through fraction or supernatant, e.g., the supernatant of the prothrombin complex centrifugation, described supra, can be further processed to isolate additional proteins, such as albumin.

An exemplary method involves sequential anion exchange chromatography steps at two distinct pH values. Prior to the first anionic exchange, the pH of the supernatant (100 mM acetate) is adjusted from pH 5.3 to pH 4.6 using 6 N HCl to remove residual charge. An Asahi Q500 anion exchange resin (cat#19907) is used, and the binding and elution parameters can be optimized for preferred albumin yield. In the first anionic exchange step, the flow-through fraction contains the albumin product and is retained for further processing. The net weight of the solution is notated and a sample is taken for testing and retain. The eluate is discarded.

Next, the pH of the flow-through fraction is adjusted to 7.4 with Tris, and the anionic exchange chromatography process is repeated. The flow-through resulting from the second anionic exchange is discarded. The albumin is eluted using 0.25 M NaCl.

The salt concentration of the albumin eluate can be reduced by UFDF according to known methods. The UFDF system can reduce the conductivity from approximately 20 mS/cm to between 7 and 8 with diafiltration against 5 volumes of buffer according to USP standards. For example, a solution having a concentration of 10% albumin contains 8 mM Na caprylate, 8 mM Na acetyl D-L tryptophan, 20 mM Tris-HCl, and 70 mM NaCl, pH 7.4. The content of the buffer (e.g., concentration of Na caprylate) will depend on the concentration of albumin in solution according to known parameters.

A sterility filtration/fill step can be performed according to methods known in the art or described herein. In one exemplary method, the albumin solution is passed through a 0.2 μm sterile filter and transferred into the Filling Suite. Approximately 100 ml of sterile bulk is put into 100 ml critically-clean, sterile glass serum vials. The albumin is pasteurized, e.g., at 60° C. for 10 hours. Vials containing the final albumin product are sealed and labeled.

### Double Salt Precipitation

The invention features alternative methods to those described in detail above. In particular, a double salt precipitation process can be performed. The only difference between the single salt precipitation process and the double salt precipitation process are two different salt concentrations during the Pooling unit operation. The premise of the two salt precipitations is that the first precipitation salts out proteins (e.g., Fg) and others but does not precipitate the immunoglobulins (e.g., IgG). The second precipitation salts out all of the immunoglobulins (including others that also salt out). The net result is that the immunoglobulins salt cut is approximately 50% pure. The Fg purification train would include the dissolution of the first paste, the S/D treatment, and the second KAcet precipitation as outlined above. The Fg product line would have the same protein profile as the second precipitation has the same parameters as the single salt process described previously. A non-limiting exemplary method of the double salt precipitation process is described below.

### First Fg Precipitation

The temperature of the pooled plasma is dropped to 20° C. and Sodium Citrate (50% citrate solution pH=7.00) is added until the final concentration is 12%. The precipitation is allowed to complete by stirring the suspension for not less than 60 minutes. The suspension is centrifuged at 5,000-20,000×G with a residence time of at least 10 minutes in the bowl (note: bowl rotation speed depends upon pool scale). The clarified solution (Supernatant #1, Super 1) is decanted and the paste is harvested (Paste #1). Super 1 is transferred into a new bag in the water-jacket vessel. The net weight of the solution is notated and a sample is taken for testing and retain.

The paste 1 is suspended as described above and taken into the S/D treatment with subsequent second KAcet precipitation. The remaining Fg product line is similar to that described above.

### Second Precipitation

The temperature of the 25 L vessel is dropped to 4° C. (+/−3° C.) and stirred appropriately for homogeneity. Additional citrate solution is added until the final citrate concentration reaches 22%. This suspension is allowed to complete precipitation by holding at 4° C. overnight at appropriate agitation. Immunoglobulins will precipitate out of solution in this 22% salt solution. The suspension is centrifuged at 5,000-20,000×G with a residence time of at least 10 minutes in the bowl (note: bowl rotation speed depends upon pool scale). The clarified solution (Supernatant #2, Super 2) is decanted and the paste is harvested (Paste #2). Super 2 is transferred into a new bag in the water-jacket vessel. The net weight of the solution is notated and a sample is taken for testing and retain. A bioburden filtration allows the Super 2 to pass into Zone 1 where the UDFD unit operation continues, e.g., into the IgG Product Line.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A process for producing a fibrinogen (Fg)-depleted preparation enriched in immunoglobulin G (IgG), comprising the steps of:
(a) contacting Fg-depleted plasma with an affinity chromatography adsorbing agent that binds IgG to produce adsorbed IgG and a solution comprising non-adsorbed proteins;
(b) separating the solution comprising non-adsorbed proteins from the adsorbed IgG; and
(c) eluting the adsorbed IgG from the adsorbing agent to produce a Fg-depleted preparation enriched in IgG.

2. The process of claim 1, wherein the affinity chromatography adsorbing agent binds to a CH3 domain of IgG.

3. The process of claim 1 or claim 2, further comprising the steps of:
(d) diafiltering the Fg-depleted preparation of enriched IgG of step (c) to produce a diafiltrate; and
(e) sterilizing the diafiltrate of step (f).

4. The process of claim 1 or 2, further comprising the steps of:
(d) passing the Fg-depleted preparation enriched in IgG of step (c) through one or more ion exchange columns to separate the IgG from contaminates; and
(e) recovering the IgG from the ion exchange column to produce a purified Fg-depleted preparation enriched in IgG.

5. The process of claim 4, wherein a portion of the contaminates bind the column.

6. The process of claim 4, wherein a portion of the IgG binds the column.

7. The process of any one of claims 4-6, wherein the ion exchange resin is a cation exchange resin.

8. The process of any one of claims 4-6, wherein the ion exchange resin is an anion exchange resin.

9. The process of any one of claims 4-8, further comprising the steps of:
(f) diafiltering the purified Fg-depleted preparation of enriched IgG of step (e) to produce a diafiltrate; and
(g) sterilizing the diafiltrate of step (f).

10. A process for producing a Fg-depleted preparation enriched in alpha-1 proteinase inhibitor (A1PI), comprising the steps of:
(a) contacting Fg-depleted plasma with an affinity chromatography adsorbing agent that binds A1PI to produce adsorbed A1PI and a solution comprising non-adsorbed proteins;
(b) separating the solution comprising non-adsorbed proteins from the adsorbed A1PI; and
(c) eluting the adsorbed A1PI from the adsorbing agent to produce a Fg-depleted preparation enriched in A1PI.

11. The process of claim 10, further comprising the steps of:
(d) diafiltering the Fg-depleted preparation of enriched A1PI of step (c) to produce a diafiltrate; and
(e) sterilizing the diafiltrate of step (d) to produce a sterilize composition.

12. The process of claim 10, further comprising the steps of:
(d) passing the Fg-depleted preparation enriched in A1PI of step (c) through one or more ion exchange columns to separate the A1PI from contaminates: and
(e) recovering the A1PI from the ion exchange column to produce a purified Fg-depleted preparation enriched in A1PI.

13. The process of claim 12, wherein a portion of the contaminates bind the column.

14. The process of claim 12, wherein a portion of the A1PI binds the column.

15. The process of any one of claims 12-14, wherein the ion exchange resin is a cation exchange resin.

16. The process of any one of claims 12-14, wherein the ion exchange resin is an anion exchange resin.

17. The process of any one of claims 12-16, further comprising the steps of:
(f) diafiltering the purified Fg-depleted preparation of enriched A1PI of step (e) to produce a diafiltrate; and
(g) sterilizing the diafiltrate of step (f) to produce a sterilized composition.

18. A process for producing a Fg-depleted preparation enriched in prothrombin complex, comprising the steps of:
(a) precipitating proteins comprising prothrombin from a Fg-depleted plasma preparation by reducing the pH to a sufficient level to produce a prothrombin complex precipitate and a solution comprising non-precipitated proteins;
(b) separating the prothrombin complex precipitate from the supernatant comprising the non-precipitated proteins;
(c) dissolving the prothrombin complex precipitate to produce a Fg-depleted preparation enriched in prothrombin complex.

19. The process of claim 18, further comprising the step of:
(d) depth filtering the Fg-depleted preparation of enriched prothrombin complex of step (c) to produce a filtrate.

20. The process of claim 19, further comprising the steps of:
(e) passing the filtrate of step (d) through one or more ion exchange columns to separate the prothrombin from contaminates; and
(f) recovering the prothrombin complex from the ion exchange column to produce a purified Fg-depleted preparation enriched in prothrombin complex.

21. The process of claim 20, wherein a portion of the contaminates bind the column.

22. The process of claim 20, wherein a portion of the prothrombin complex binds the column.

23. The process of any one of claims 20-22, wherein the ion exchange resin is an anion exchange resin.

24. The process of any one of claims 20-22, wherein the ion exchange resin is a cation exchange resin.

25. The process of any one of claims 20-24, further comprising the steps of:
(g) diafiltering the purified Fg-depleted preparation of enriched prothrombin complex of step (f) to produce a diafiltrate; and
(h) sterilizing the diafiltrate of step (g) to produce a sterilized composition.

26. A process for producing a Fg-depleted preparation enriched in albumin, comprising the steps of:
(a) passing an Fg-depleted plasma preparation through one or more anion exchange columns to separate the albumin from contaminates; and
(b) recovering the flow-through fraction containing albumin from the anion exchange column;
(c) raising the pH of the flow-through fraction containing albumin;
(d) passing the flow-through fraction containing albumin through one or more anion exchange columns to separate the albumin from contaminates; and
(e) recovering the albumin from the anion exchange column to produce a purified Fg-depleted preparation enriched in albumin.

27. The process of claim 26, further comprising the steps of:
(f) diafiltering the purified Fg-depleted preparation of albumin of step (e) to produce a diafiltrate; and
(g) sterilizing the diafiltrate of step (f) to produce a sterilized composition.

28. The process of any of claims 1-27, wherein the Fg-depleted preparation is produced by the steps of:
(a) precipitating proteins comprising Fg from whole plasma by addition of a salt in sufficient quantity to produce a Fg precipitate; and
(b) separating the Fg precipitate from the supernatant to produce a Fg-depleted preparation.

29. The process of any one of claims 1-28, wherein step (a) is preceded by diafiltering the Fg-depleted plasma.

30. The process of any of claims 1-29, wherein step (a) is preceded by virally reducing the Fg-depleted plasma.

31. The process of claim 30, wherein the virally reducing comprises incubating the plasma sample with a viricide solvent/detergent solution in an amount and duration sufficient to inactivate lipid-containing viruses.

32. A process for producing a Fg-depleted preparation enriched in a plasma protein of interest, comprising the steps of:
(a) contacting Fg-depleted plasma with an affinity chromatography adsorbing agent that binds the plasma protein of interest to produce adsorbed protein of interest and a solution comprising non-adsorbed proteins;
(b) separating the solution comprising non-adsorbed proteins from the adsorbed protein of interest; and
(c) eluting the adsorbed protein of interest from the adsorbing agent to produce a Fg-depleted preparation enriched in a protein of interest.

33. The process of claim 32, further comprising the steps of:
(d) passing the Fg-depleted preparation enriched in a protein of interest of step (c) through one or more ion exchange columns to separate the protein of interest from contaminates; and
(e) recovering the protein of interest from the ion exchange column to produce a Fg-depleted preparation enriched in a protein of interest.

34. The process of claim 33, wherein a portion of the contaminates bind the column.

35. The process of claim 33, wherein a portion of the protein of interest binds the column.

36. A process for producing a Fg-depleted preparation enriched in a first protein of interest and a Fg-depleted preparation enriched in a second protein of interest, comprising the steps of:
(a) contacting Fg-depleted plasma with a first affinity chromatography adsorbing agent that binds the first protein of interest to produce an adsorbed first protein of interest and a first solution comprising non-adsorbed proteins:
(b) separating the first solution comprising non-adsorbed proteins from the adsorbed first protein of interest;
(c) eluting the adsorbed first protein of interest from the adsorbing agent to produce a Fg-depleted preparation enriched in the first protein of interest;
(d) contacting the first solution comprising non-adsorbing proteins with a second affinity chromatography adsorbing agent that binds the second protein of interest to produce an adsorbed second protein of interest and a second solution comprising non-adsorbed proteins;
(e) separating the second solution comprising non-adsorbed proteins from the adsorbed second protein of interest; and
(f) eluting the adsorbed second protein of interest from the second adsorbing agent to produce a Fg-depleted preparation enriched in the second protein of interest.

37. The process of claim 36, further comprising the steps of:
(g) passing the preparation enriched in the first protein of interest through one or more ion exchange columns to separate the first protein of interest from contaminates; and
(h) recovering the adsorbed first protein of interest from the ion exchange resin to produce a purified Fg-depleted preparation enriched in the first protein of interest.

38. The process of claim 37, wherein a portion of the contaminates bind the column.

39. The process of claim 37, wherein a portion of the first protein of interest binds the column.

40. The process of claim 36, further comprising the steps of:
(g) passing the preparation enriched in the second protein of interest through one or more ion exchange columns; and
(h) recovering the adsorbed second protein of interest from the ion exchange column to produce a purified Fg-depleted preparation enriched in the second protein of interest.

41. The process of claim 40, wherein a portion of the contaminates bind the column.

42. The process of claim 40, wherein a portion of the second protein of interest binds the column.

43. The process of any one of claims 36-42, wherein the first plasma protein of interest is immunoglobulin G (IgG) and the second protein of interest is A1PI.

44. The process of claim 43, wherein the first affinity chromatography agent binds to a CH3 domain of IgG.

45. The process of any one of claims 36-42, wherein the first plasma protein of interest is A1PI and the second protein of interest is IgG.

46. The process of any one of claims 36-45, wherein the first ion exchange column comprises a cationic resin.

47. The process of any one of claims 36-45, wherein the second exchange column comprises an anionic resin.

48. The process of claim 47, wherein the anionic resin is a weak anionic resin.

49. A process for producing a Fg-depleted preparation enriched in a first protein of interest, a Fg-depleted preparation enriched in a second protein of interest, and an Fg-depleted preparation enriched in a third protein of interest, comprising the steps of:
(a) contacting Fg-depleted plasma with a first affinity chromatography adsorbing agent that binds the first protein of interest to produce an adsorbed first protein of interest and a first solution comprising non-adsorbed proteins:
(b) separating the first solution comprising non-adsorbed proteins from the adsorbed first protein of interest;
(c) eluting the adsorbed first protein of interest from the adsorbing agent to produce a Fg-depleted preparation enriched in the first protein of interest;
(d) contacting the first solution comprising non-adsorbing proteins with a second affinity chromatography adsorbing agent that binds the second protein of interest to produce an adsorbed second protein of interest and a second solution comprising non-adsorbed proteins;
(e) separating the second solution comprising non-adsorbed proteins from the adsorbed second protein of interest;
(f) eluting the adsorbed second protein of interest from the second adsorbing agent to produce a Fg-depleted preparation enriched in the second protein of interest;
(g) precipitating proteins comprising a third protein of interest from the second solution comprising non-adsorbed proteins by reducing the pH to a sufficient level to produce a precipitate comprising the third protein of interest and a solution comprising non-precipitated proteins;
(h) separating the precipitate comprising the third protein of interest from the supernatant comprising the non-precipitated proteins; and
(i) dissolving the precipitate comprising the third protein of interest to produce a Fg-depleted preparation enriched in the third protein of interest.

50. A process for producing a Fg-depleted preparation enriched in a first protein of interest, a Fg-depleted preparation enriched in a second protein of interest, an Fg-depleted preparation enriched in a third protein of interest, and an Fg-depleted preparation enriched in a fourth protein of interest, comprising the steps of:
(a) contacting Fg-depleted plasma with a first affinity chromatography adsorbing agent that binds the first protein of interest to produce an adsorbed first protein of interest and a first solution comprising non-adsorbed proteins:
(b) separating the first solution comprising non-adsorbed proteins from the adsorbed first protein of interest;
(c) eluting the adsorbed first protein of interest from the adsorbing agent to produce a Fg-depleted preparation enriched in the first protein of interest;
(d) contacting the first solution comprising non-adsorbing proteins with a second affinity chromatography adsorbing agent that binds the second protein of interest to produce an adsorbed second protein of interest and a second solution comprising non-adsorbed proteins;
(e) separating the second solution comprising non-adsorbed proteins from the adsorbed second protein of interest;
(f) eluting the adsorbed second protein of interest from the second adsorbing agent to produce a Fg-depleted preparation enriched in the second protein of interest;
(g) precipitating proteins comprising a third protein of interest from the second solution comprising non-adsorbed proteins by reducing the pH to a sufficient level to produce a precipitate comprising the third protein of interest and a solution comprising non-precipitated proteins;
(h) separating the precipitate comprising the third protein of interest from the supernatant comprising the non-precipitated proteins;
(i) dissolving the precipitate comprising the third protein of interest to produce a Fg-depleted preparation enriched in the third protein of interest;
(j) passing the supernatant comprising the non-precipitated proteins of step (h) through one or more anion exchange columns to separate the fourth protein of interest from contaminates;
(k) recovering the flow-through fraction containing the fourth protein of interest from the anion exchange column;
(l) raising the pH of the flow-through fraction containing the fourth protein of interest;
(m) passing the flow-through fraction containing the fourth protein of interest through one or more anion exchange columns to separate the fourth protein of interest from contaminates; and
(n) recovering the fourth protein of interest from the anion exchange column to produce a purified Fg-depleted preparation enriched in the fourth protein of interest.

51. The process of claim 49 or 50, wherein the third protein of interest is prothrombin complex.

52. The process of claim 50, wherein the fourth protein of interest is albumin.

53. A process for producing a Fg-depleted preparation enriched in a plasma protein of interest, comprising the steps of:
(a) precipitating proteins comprising Fg from whole plasma by addition of a salt in sufficient quantity to produce a Fg precipitate and a solution comprising a plasma protein of interest;
(b) separating the Fg precipitate from the supernatant comprising the plasma protein of interest;
(c) diafiltering the supernatant comprising the plasma protein of interest to produce a solution;
(d) contacting the solution of step (c) with an affinity chromatography adsorbing agent that binds the plasma protein of interest to produce adsorbed protein of interest and a solution comprising non-adsorbed proteins;
(e) separating a solution comprising non-adsorbed proteins from the adsorbed protein of interest; and
(f) eluting the adsorbed protein of interest from the adsorbing agent to produce a Fg-depleted preparation enriched in a protein of interest.

54. The process of claim 53, wherein the Fg precipitate comprises plasminogen.

55. The process of claim 53, wherein the Fg precipitate is substantially free of plasminogen.

56. The process of claim 53, wherein the Fg-depleted preparation of step (f) comprises at least 30% of the protein of interest from the whole plasma.

57. The process of claim 53, wherein the Fg precipitate comprises at least 30% of an the Fg from the whole plasma

58. The process of any one of claims 53-57, further comprising the steps of:
(g) passing the Fg-depleted preparation enriched in a protein of interest of step (c) through one or more ion exchange columns to separate the protein of interest from contaminates; and
(h) recovering adsorbed protein of interest from the ion exchange column to produce a purified Fg-depleted preparation enriched in a protein of interest.

59. The process of claim 58, wherein a portion of the contaminates bind the column.

60. The process of claim 58, wherein a portion of the protein of interest binds the column.

61. The process of any one of claims 58-60, wherein the protein of interest is IgG.

62. The process of any one of claims 39-46, wherein the protein of interest is A1PI.

63. The process of any one of claims 39-46, wherein the protein of interest is prothrombin complex or albumin.

64. The process of any one of claims 53-63, wherein the ion exchange resin comprises a cationic resin.

65. The process of any one of claims 53-63, wherein the ion exchange resin comprises an anionic resin.

66. The process of any one of claims 53-65, further comprising the steps of:
(h) reconstituting the Fg precipitate in a suitable buffer to produce a Fg solution;
(i) incubating the Fg solution with a viricide solvent/detergent solution in an amount sufficient to inactivate lipid-containing viruses;
(j) precipitating proteins comprising Fg from the solution resulting from step (i) by addition of a salt in sufficient quantity to achieve a precipitation of Fg; and
(k) recovering the precipitate of step (i).

67. The process of claim 66, further comprising the steps of:
(l) diafiltering the precipitate of step (h) to produce a diafiltrate; and
(m) sterilizing the diafiltrate of step (I) to produce a sterilized diafiltrate.

68. A process for producing a Fg-depleted preparation enriched in a first plasma protein of interest and a Fg-depleted preparation enriched in a second plasma protein of interest, comprising the steps of:
(a) precipitating proteins comprising Fg from whole plasma by addition of a salt in sufficient quantity to produce a Fg precipitate and a supernatant comprising the first plasma protein of interest and the second plasma protein of interest;
(b) separating the Fg precipitate from the supernatant;
(c) diafiltering the supernatant until a solution obtained;
(d) contacting Fg-depleted plasma with a first affinity chromatography adsorbing agent that binds the first plasma protein of interest to produce an adsorbed first protein of interest and a first solution comprising non-adsorbed proteins:
(e) separating the first solution comprising non-adsorbed proteins from the adsorbed first protein of interest;
(f) eluting the adsorbed first protein of interest from the adsorbing agent to produce a Fg-depleted preparation enriched in the first plasma protein of interest;
(g) contacting the first solution comprising non-adsorbing proteins with a second affinity chromatography adsorbing agent that binds the second plasma protein of interest to produce an adsorbed second protein of interest and a second solution comprising non-adsorbed proteins;
(h) separating the second solution comprising non-adsorbed proteins from the adsorbed second protein of interest; and
(i) eluting the adsorbed second protein of interest from the second adsorbing agent to produce a Fg-depleted preparation enriched in the second plasma protein of interest.

69. The process of claim 68, wherein the Fg precipitate comprises plasminogen.

70. The process of claim 68, wherein the Fg precipitate is substantially free of plasminogen.

71. The process of any one of claims 68-70, further comprising the steps of:
(j) passing the preparation enriched in the first protein of interest through one or more ion exchange columns to separate the first protein of interest from contaminates; and
(k) recovering the first protein of interest from the ion exchange column to produce a purified Fg-depleted preparation enriched in the first protein of interest.

72. The process of claim 71, wherein a portion of the contaminates bind the column.

73. The process of claim 71, wherein a portion of the first protein of interest binds the column.

74. The process of any one of claims 68-73, further comprising the steps of:
(j) passing the preparation enriched in the second protein of interest through one or more ion exchange columns to separate the second protein of interest from contaminates; and
(k) recovering the second protein of interest from the ion exchange column to produce a purified Fg-depleted preparation enriched in the second protein of interest.

75. The process of claim 74, wherein a portion of the contaminates bind the column.

76. The process of claim 74, wherein a portion of the second protein of interest binds the column.

77. The process of any one of claims 68-76, wherein the first protein of interest is IgG and the second protein of interest is A1PI.

78. The process of any one of claims 68-76, wherein the first protein of interest is A1PI and the second protein of interest is IgG.

79. The process of claim 77 or 78, wherein the first affinity chromatography agent binds to a CH3 domain of IgG.

80. The process of any one of claims 68-79, wherein the first ion exchange column comprises a cationic resin.

81. The process of any one of claims 68-80, wherein the second exchange column comprises an anionic resin.

82. The process of claim 81, wherein the anionic resin is a weak anionic resin.

83. A process for producing a Fg-depleted preparation enriched in a first plasma protein of interest, a Fg-depleted preparation enriched in a second plasma protein of interest, and a Fg-depleted preparation enriched in a third plasma protein of interest, comprising the steps of:
(a) precipitating proteins comprising Fg from whole plasma by addition of a salt in sufficient quantity to produce a Fg precipitate and a supernatant comprising the first plasma protein of interest and the second plasma protein of interest;
(b) separating the Fg precipitate from the supernatant;
(c) diafiltering the supernatant until a solution obtained;
(d) contacting Fg-depleted plasma with a first affinity chromatography adsorbing agent that binds the first plasma protein of interest to produce an adsorbed first protein of interest and a first solution comprising non-adsorbed proteins:
(e) separating the first solution comprising non-adsorbed proteins from the adsorbed first protein of interest;
(f) eluting the adsorbed first protein of interest from the adsorbing agent to produce a Fg-depleted preparation enriched in the first plasma protein of interest;
(g) contacting the first solution comprising non-adsorbing proteins with a second affinity chromatography adsorbing agent that binds the second plasma protein of interest to produce an adsorbed second protein of interest and a second solution comprising non-adsorbed proteins;
(h) separating the second solution comprising non-adsorbed proteins from the adsorbed second protein of interest; and
(i) eluting the adsorbed second protein of interest from the second adsorbing agent to produce a Fg-depleted preparation enriched in the second plasma protein of interest;
(j) precipitating proteins comprising a third protein of interest from the second solution comprising non-adsorbed proteins by reducing the pH to a sufficient level to produce a precipitate comprising the third protein of interest and a solution comprising non-precipitated proteins;
(k) separating the precipitate comprising the third protein of interest from the supernatant comprising the non-precipitated proteins; and
(l) dissolving the precipitate comprising the third protein of interest to produce a Fg-depleted preparation enriched in the third protein of interest.

84. A process for producing a Fg-depleted preparation enriched in a first plasma protein of interest, a Fg-depleted preparation enriched in a second plasma protein of interest, a Fg-depleted preparation enriched in a third plasma protein of interest, and a Fg-depleted preparation enriched in a fourth plasma protein of interest, comprising the steps of:
(a) precipitating proteins comprising Fg from whole plasma by addition of a salt in sufficient quantity to produce a Fg precipitate and a supernatant comprising the first plasma protein of interest and the second plasma protein of interest;
(b) separating the Fg precipitate from the supernatant;
(c) diafiltering the supernatant until a solution obtained;
(d) contacting Fg-depleted plasma with a first affinity chromatography adsorbing agent that binds the first plasma protein of interest to produce an adsorbed first protein of interest and a first solution comprising non-adsorbed proteins:
(e) separating the first solution comprising non-adsorbed proteins from the adsorbed first protein of interest;
(f) eluting the adsorbed first protein of interest from the adsorbing agent to produce a Fg-depleted preparation enriched in the first plasma protein of interest;
(g) contacting the first solution comprising non-adsorbing proteins with a second affinity chromatography adsorbing agent that binds the second plasma protein of interest to produce an adsorbed second protein of interest and a second solution comprising non-adsorbed proteins;
(h) separating the second solution comprising non-adsorbed proteins from the adsorbed second protein of interest; and
(i) eluting the adsorbed second protein of interest from the second adsorbing agent to produce a Fg-depleted preparation enriched in the second plasma protein of interest;
(j) precipitating proteins comprising a third protein of interest from the second solution comprising non-adsorbed proteins by reducing the pH to a sufficient level to produce a precipitate comprising the third protein of interest and a solution comprising non-precipitated proteins;
(k) separating the precipitate comprising the third protein of interest from the supernatant comprising the non-precipitated proteins;
(l) dissolving the precipitate comprising the third protein of interest to produce a Fg-depleted preparation enriched in the third protein of interest;
(m) passing the supernatant comprising the non-precipitated proteins of step (l) through one or more anion exchange columns to separate the fourth protein of interest from contaminates;
(n) recovering the flow-through fraction containing the fourth protein of interest from the anion exchange column;
(o) raising the pH of the flow-through fraction containing the fourth protein of interest;
(p) passing the flow-through fraction containing the fourth protein of interest through one or more anion exchange columns to separate the fourth protein of interest from contaminates; and
(q) recovering the fourth protein of interest from the anion exchange column to produce a purified Fg-depleted preparation enriched in the fourth protein of interest.

85. The process of claim 83 or 84, wherein the third protein of interest is prothrombin complex.

86. The process of claim 84, wherein the fourth protein of interest is albumin.

87. The process of any one of claims 68-86, further comprising the steps of:
(l) reconstituting the Fg precipitate in a suitable buffer to produce a Fg solution;
(m) incubating the Fg solution with a viricide solvent/detergent solution in an amount sufficient to inactivate lipid-containing viruses;
(n) precipitating proteins comprising Fg from the solution of step (l) by addition of a salt to produce a Fg precipitate; and
(o) recovering the precipitate of step (n).

88. The process of claim 87, further comprising the steps of:
(p) diafiltering the precipitate of step (n) to produce a diafiltrate; and
(q) sterilizing the diafiltrate of step (p) to produce a sterilized diafiltrate.

89. A process for producing a Fg-depleted preparation enriched in a plasma protein of interest, comprising the steps of:
(a) precipitating proteins comprising Fg from whole plasma by addition of a first salt in sufficient quantity to produce a Fg precipitate and a supernatant comprising the protein of interest;
(b) precipitating proteins comprising the plasma protein of interest from the supernatant by addition of second salt in sufficient quantity to produce a precipitate comprising the protein of interest;
(c) reconstituting the precipitate in a suitable buffer to produce a solution;
(d) contacting the solution of step (c) with an affinity chromatography adsorbing agent that binds the protein of interest to produce adsorbed protein of interest and a solution comprising non-adsorbed proteins;
(e) separating the solution comprising non-adsorbed proteins from the adsorbed protein of interest; and
(f) eluting the adsorbed protein of interest from the adsorbing agent to produce a Fg-depleted preparation enriched in the protein of interest.

90. The process of any one of claims 53-89, wherein the salt comprises potassium acetate.

91. The process of any one of claims 53-89, wherein the salt comprises trisodium citrate.

92. The process of any one of claims 1-91, further comprising diluting a solution to reduce a salt concentration of the solution.

93. The process of any one of claim 32-35, 53-67, or 89, wherein the plasma protein of interest comprises antithrombin III, fibronectin, plasminogen, Factor VII, Factor VIII, Factor IX, Factor X, Factor XI, or Factor XIII.

94. The process of any one of claim 36-52 or 68-88, wherein one or both of the first and second plasma protein of interest is antithrombin III, fibronectin, plasminogen, Factor VII, Factor VIII, Factor IX, Factor X, Factor XI, or Factor XIII.
